(19) United States
(12) Patent Application Publication
(10) Pub. No.: US 2015/0305913 A1
(43) Pub. Date: Oct. 29, 2015

(54) SUPPORT DEVICES

(71) Applicants: Deborah Calegory, St. George, UT (US); Jeffrey Calegory, Santa Clara, UT (US); Tom A. Calegory, St. George, UT (US)

(72) Inventors: Deborah Calegory, St. George, UT (US); Jeffrey Calegory, Santa Clara, UT (US); Tom A. Calegory, St. George, UT (US)

(21) Appl. No.: 14/698,594

(22) Filed: Apr. 28, 2015

Related U.S. Application Data
(60) Provisional application No. 61/985,595, filed on Apr. 29, 2014.

(51) Int. Cl. A61F 5/055

(52) U.S. Cl. CPC A61F 5/055

(57) ABSTRACT

The present invention is directed to head support device. A device may include a first portion configured to be positioned adjacent a portion of the head of the user and a second portion configured to be positioned adjacent a chest area of the user. The device may further include a third portion extending between the first portion and the second portion and including an angular section.

### CROSS-REFERENCE TO RELATED APPLICATION

A claim for the benefit of priority to the Apr. 29, 2014 filing date of U.S. Provisional Patent Application No. 61/985,595, titled HEAD BOB (“the '585 Provisional Application”), is hereby made pursuant to 35 U.S.C. §119(e). The entire disclosure of the '595 Provisional Application is, hereby incorporated herein.

### TECHNICAL FIELD

This disclosure relates generally to support devices and, more specifically, to support devices for stabilizing a user's head in an upright position.

### BRIEF SUMMARY OF THE INVENTION

In one specific embodiment, a device includes a first portion configured to be positioned adjacent a portion of a head of a user. In one embodiment, the first portion comprises a “u-shaped” portion. The device may further include a second portion configured to be positioned adjacent a chest area of the user. In addition, the device may include a third portion extending between the first portion and the second portion and including an angular section.

In another specific embodiment, a device, for stabilizing a head of a user in an upright position, includes a chin support comprising a substantially u-shaped portion configured to contact a portion of a chin area of the user. The device may further include a first portion extending from the chin support and a second portion extending from and at an angle to the first portion. Moreover, the device includes a chest portion extending from the second portion and configured to contact a chest area of the user.

According to another embodiment, a device includes a unitary piece of material configured to support a head of a user. The device may include a first portion configured to be positioned adjacent a portion of the head of the user. The device may also include a second portion configured to be positioned adjacent a chest area of the user. Further, the device may include a third portion extending between the first portion and the second portion and including an angular section.

Other aspects, as well as features and advantages of various aspects, of the present invention will become apparent to those of skill in the art through consideration of the ensuing description, the accompanying drawings and the appended claims.

### BRIEF DESCRIPTION OF THE DRAWINGS

FIGS. 1-4 depict a support device, in accordance with various embodiments of the present disclosure.

FIG. 5 is another depiction of the support device, according to an embodiment of the present disclosure.

FIGS. 6-8 depict the support device of FIGS. 1-5 being used by a user.

FIGS. 9 and 10 are addition illustrations of the support device, in accordance with embodiments of the present disclosure.

### DETAILED DESCRIPTION

Referring in general to the accompanying drawings, various embodiments of the present invention are illustrated to show the structure for support devices. Common elements of the illustrated embodiments are designated with like numerals. It should be understood that the figures presented are not meant to be illustrative of actual views of any particular portion of the actual device structure, but are merely schematic representations which are employed to more clearly and fully depict embodiments of the invention.

The following provides a more detailed description of the present invention and various representative embodiments thereof. In this description, functions may be shown in block diagram form in order not to obscure the present invention in unnecessary detail. Additionally, block definitions and partitioning of logic between various blocks is exemplary of a specific implementation. It will be readily apparent to one of ordinary skill in the art that the present invention may be practiced by numerous other partitioning solutions. For the most part, details concerning timing considerations and the like have been omitted where such details are not necessary to obtain a complete understanding of the present invention and are within the abilities of persons of ordinary skill in the relevant art.

Embodiments of the present disclosure include a device configured to stabilize a user's head in an upright position. In one embodiment, the device may include a single piece of material (e.g., plastic) with no welds or seams. By way of example only, the device may be made at least partially from a computer numerical control (CNC) machine. It is noted that the device may comprise any suitable material. As an example, the device may comprise plastic (e.g., thermoset plastic, thermoplastic, or a combination thereof). In a more specific embodiment, the device may comprise a relatively hard plastic. It is further noted that the device may comprise a unitary piece of material, such as a unitary piece of plastic. Further, in various embodiments, the device may not require moving parts.

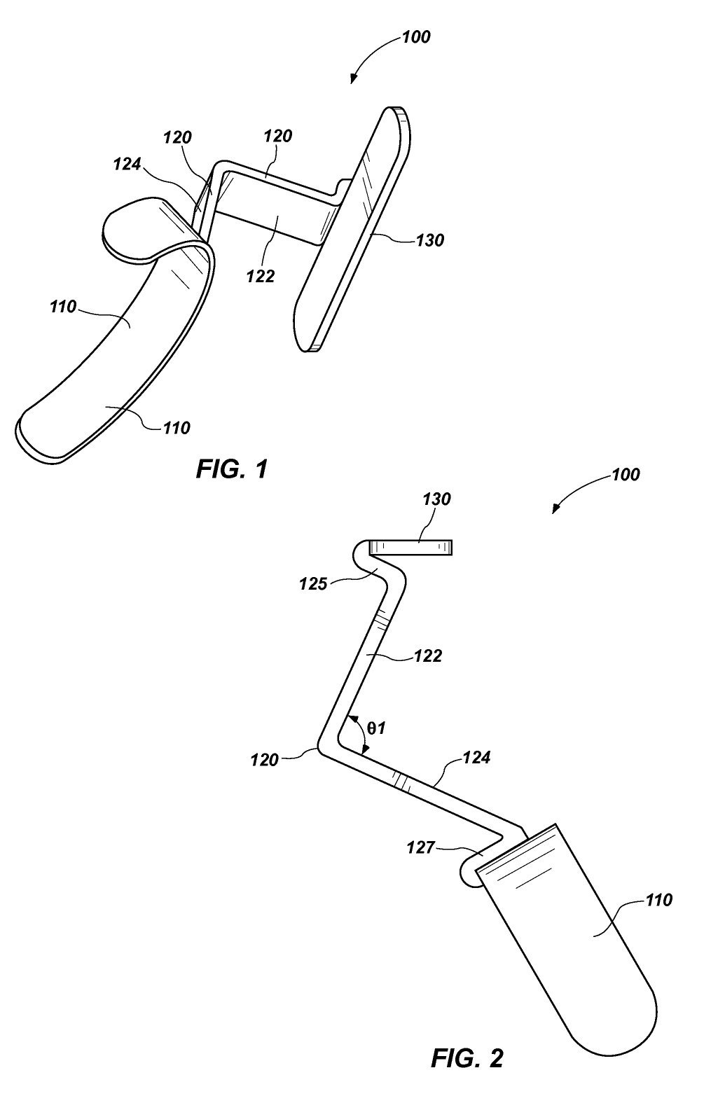

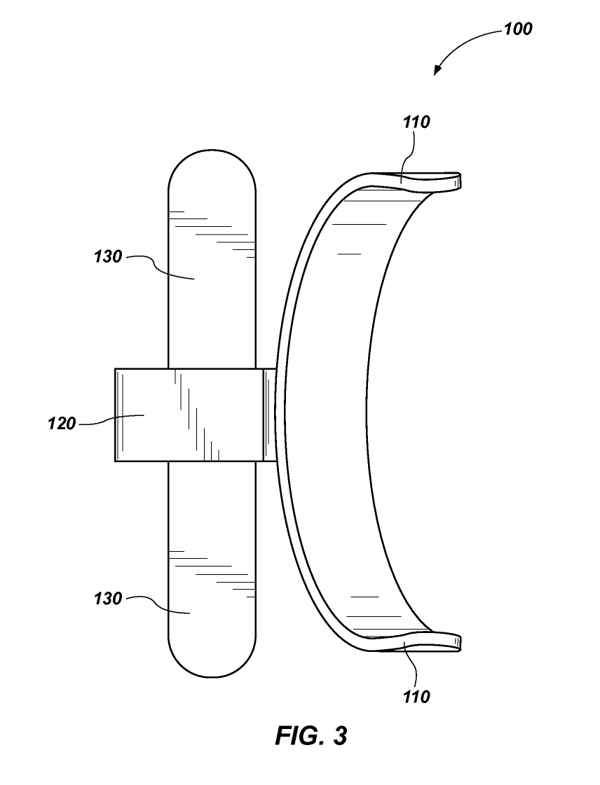

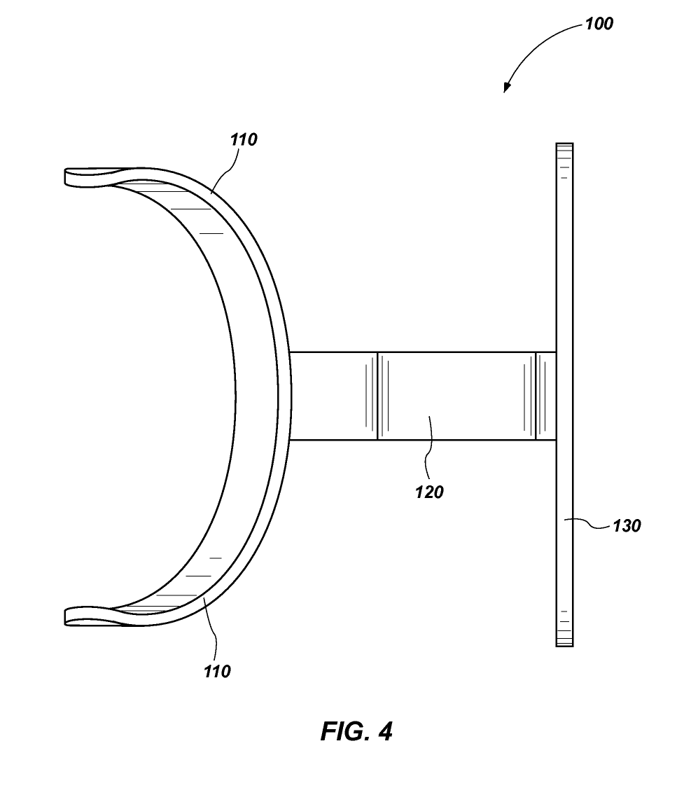

FIGS. 1-4 illustrate a device 100, in accordance with various embodiments of the present disclosure. As illustrated in FIGS. 1-4, device 100 includes a portion 110, a portion 120, and a portion 130. Portion 110, which may also be referred to herein as a “u-shaped portion” or a “chin support,” is configured to receive a portion of a head of a user. More specifically, in one example, portion 110 is configured to be positioned adjacent to and in contact with a portion of the user's head. In a more specific example, a user may position his or her jaw, and possibly his or her chin, in contact with portion 110. It is noted that a size of portion 110 may vary to provide suitable stability and comfort for various sized users. Further, in one embodiment, a strip of material (e.g., a rubber strip), which, for example, may comprise a pad (e.g., a padded cushion), may be positioned adjacent to and in contact with portion 110. As an example, the strip of material may be affixed to portion 110 via an adhesive. In one non-limiting example, the strip of material may comprise a neoprene strip. As will be appreciated by a person having ordinary skill in the art, the strip may provide a cushion (i.e., padding) to a user.

Portion 120, which may also be referred to herein as a “angular portion,” extends from portion 110 and includes a first piece 122 and a second piece 124, which is at an angle Θ₁ with respect to first piece 122. First piece 122 may extend from portion 130 and second piece 124 may extend from portion 110. It is noted that although angle Θ₁ between first piece 122 and second piece is depicted as being approximately 90 degrees, the present disclosure is not so limited. Rather, angle Θ₁ may comprise any suitable sized angle, and angle Θ₁ may vary depending on a size of device 100. Portion 120 may also include a third piece 125, which extends from and is at an angle relative to first piece 122. In one non-limiting example, third piece 125 is substantially perpendicular to piece 122. Portion 120 may also include a fourth piece 127, which extends from and is at an angle relative to second piece 124. In one non-limiting example, an angle between fourth piece 127 and second piece 124 is an acute angle. Further, one surface of fourth piece 127 may abut a surface of portion 130.

Portion 130, which may also be referred to herein as a “chest portion” is configured to be positioned adjacent to and in contact with a portion of the user's chest. Stated another way, portion 130 may be configured for resting on the user's chest, and may provide support and help keep device 100 in a desired position. As illustrated in at least FIG. 2, portion 130 is at an angle to and extends from piece 125 of portion 120. In one non-limiting example, the angle between piece 125 and portion 130 is an acute angle. Further, one surface of fourth piece 127 may abut a surface of portion 110. According to one embodiment, at least one side of portion 130 may be substantially planar (i.e., for contacting a user's chest area). Further, it is noted that a size and shape of portion 130 may vary to provide suitable stability and comfort.

Further, the bottom portion 130 may include a strip of material (e.g., a rubber material) that may act as a non-sticky adhesive on bottom portion 130 and may aid to keep bottom portion 130 comfortable for a user and in place whether bottom portion 130 is placed on the user's skin or clothing. In one example, the strip may be affixed to bottom portion 130 via any suitable manner, such as an adhesive. The strip of material may comprise, for example only, a synthetic rubber, such as a neoprene strip. According to one embodiment, the strip of material may comprise a pad (e.g., a cushioned pad), which may provide a cushion (i.e., padding) to a user.

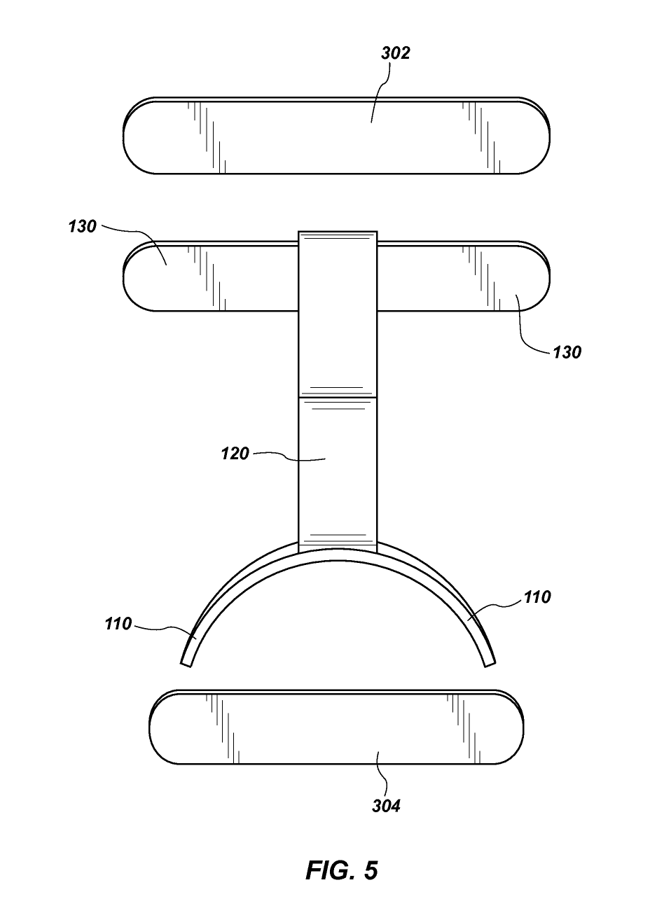

FIG. 5 illustrates device 100 including a strip 302 configured to be positioned adjacent a surface of portion 130. As will be understood, strip 302, which may comprise, for example only, a material (e.g., a rubber material, such as neoprene), may be affixed (e.g. via an adhesive) to a surface of portion 130 and may be positioned adjacent to and in contact with a user (e.g., in contact with a user's chest) during use of device 100. Stated another way, strip 302 may be configured to be positioned between a surface of portion 130 and a user. Strip 302, which may also be referred to as a “pad,” may provide a cushion (i.e., padding) to a user.

FIG. 5 further illustrates a strip 304 configured to be positioned adjacent a surface of portion 110. As will be understood, strip 304, which may comprise a piece of material (e.g., a rubber material, such as neoprene), may be affixed (e.g., via an adhesive) to a surface of portion 110 and may be positioned adjacent to an in contact with a user (e.g., in contact with a user's chin and/or jaw) during use of device 100. Stated another way, strip 304 may be configured to be positioned between a surface of portion 110 and a user. Strip 304, which may also be referred to as a “pad,” may provide a cushion (i.e., padding) to a user. According to one embodiment, each of strip 302 and strip 304 may be removable and/or replaceable. Further, each of strip 302 and strip 304 may be wiped clean, either after being removed or while being affixed to portion 110. Further, each of strip 302 and strip 304 may be removed and, thereafter, new strips may be positioned on device 100.

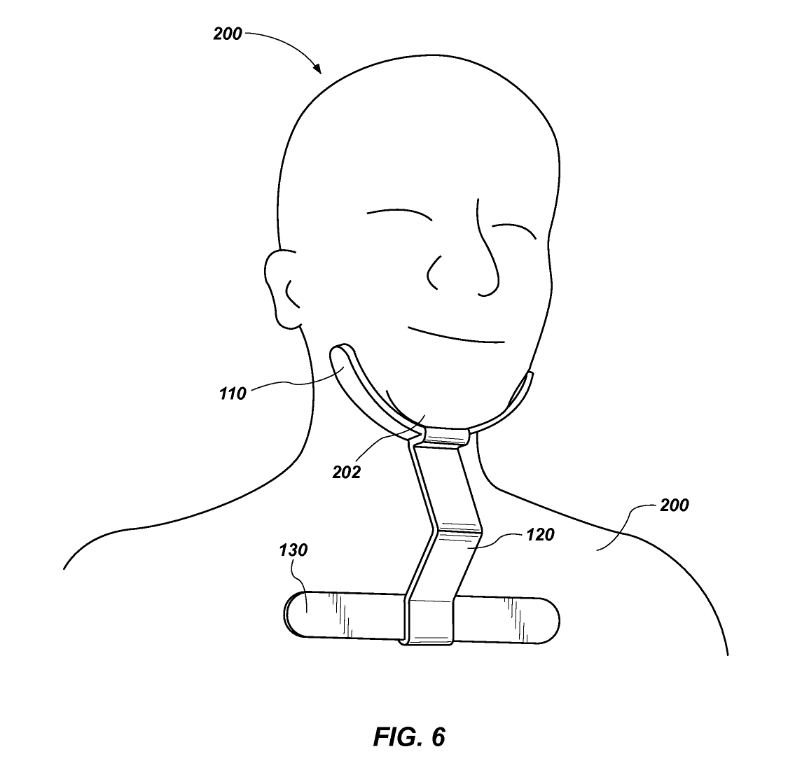

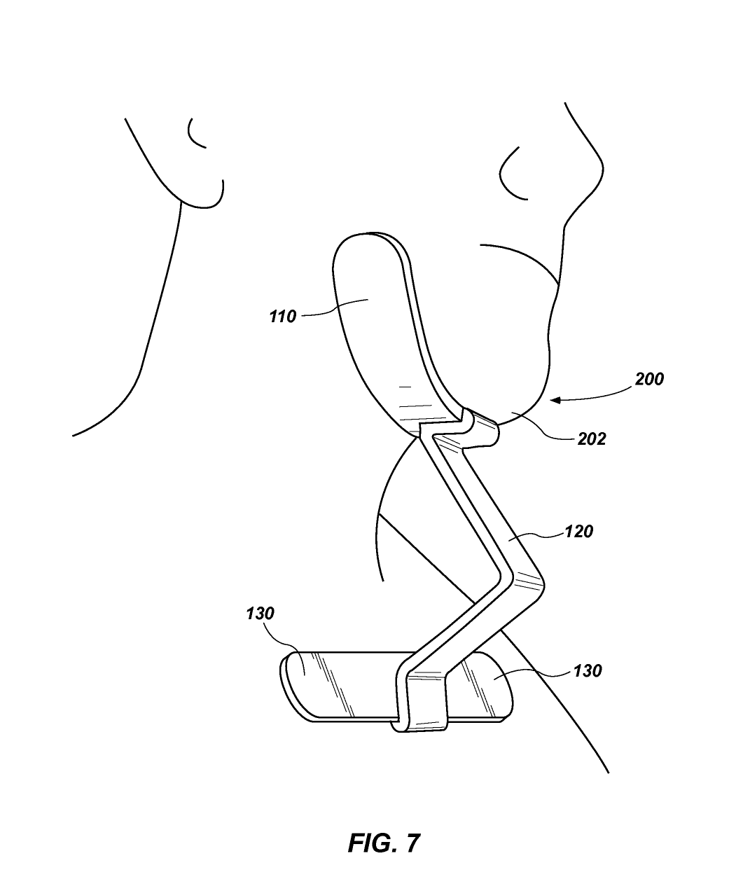

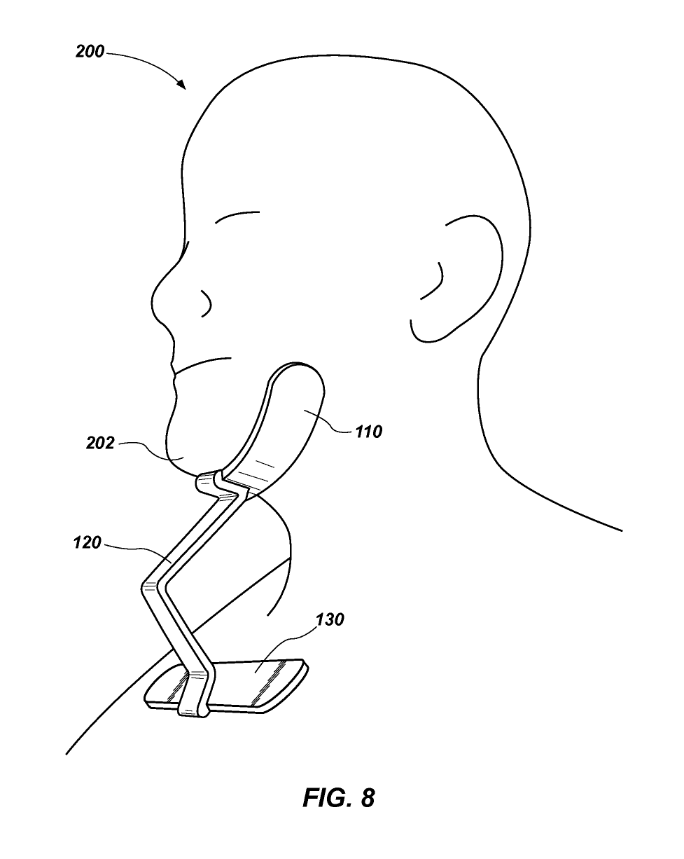

With reference to FIGS. 6-8, which depict device 100 “in use” by a user 200, portion 110 is positioned adjacent to and in contact with a jaw 202 of user 200. Further, portion 130 is positioned adjacent to an in contact with a chest area of user 200. It is noted that it is noted required for either portion 110 or portion 130 to be in contact with the skin of a user. It is further noted that a position of device 200 relative to a user (e.g., user 200) may be adjusted based on a desire of user (e.g., for comfort). More specifically, a position of portion 110 and/or portion 130 relative to a user may be adjusted.

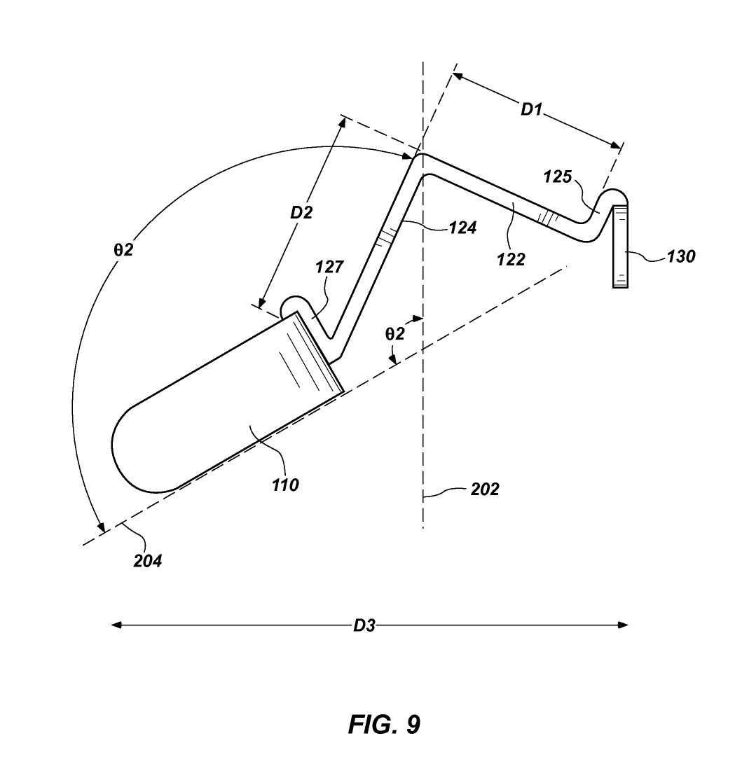

FIG. 9 is another depiction of device 100. As illustrated in FIG. 9, a dimension D1 of first piece 122 may comprise, as a non-limiting example, 1.812 inches, and a dimension D2 of second piece 124 may comprise, as a non-limiting example, 2.306 inches. Further, a dimension D3, which extends from portion 130 to portion 110, may comprise, for example only, 6.060 inches. Further, an angle Θ₂ (i.e., between an axis 402 extending through a corner of portion 120 (and parallel to a longitudinal axis of portion 130) and an axis 404 parallel to an surface of portion 110), may comprise, as a non-limiting example, 145 degrees.

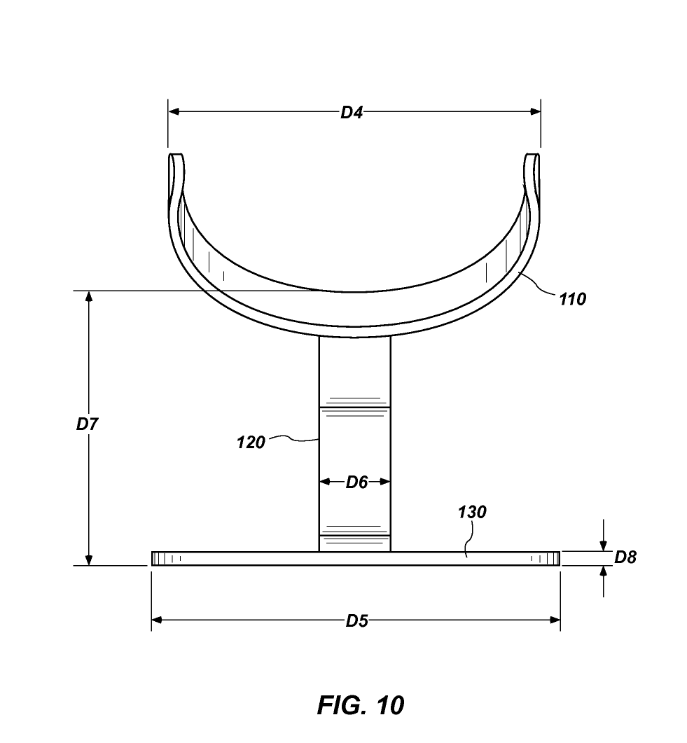

Further, with reference to FIG. 10, which is another depiction of device 100, a dimension D4 may comprise, for example only, 5.259 inches, a dimension D5 may comprise, for example only, 6.062 inches, a dimension D6 may comprise, for example only, 1.062 inches, and a dimension D7 may comprise, for example only, 4.134 inches. It is noted that the dimensions depicted and disclosed with reference to FIGS. 9 and 10 are provided only as examples, and the dimensions may vary in any suitable manner.

As will be appreciated by a person having ordinary skill in the art, a device 100 may be used as a traveler's aid (e.g., in a plane, train or automobile) for resting and sleeping. Device 100 may be used an alternative to a travel pillow and may be much easier to use and carry. Device 100 may also be used anywhere a user wants to catch some rest (e.g., emergency waiting room, church, at home watching television, etc.). Device 100, and more specifically, portion 110, may be configured to help keep a user's mouth in a substantially closed position (e.g., to prevent drooling). The user's head may stay upright, which may help prevent a kinked neck and/or infringement on fellow passengers (e.g., when the user's head leans too far to one side or the other). It is noted that in contrast to some conventional devices, device 100 does not require a back strap (i.e., a strap which wraps around a portion of a user's head).

Although the foregoing description contains many specifics, these should not be construed as limiting the scope of the invention or of any of the appended claims, but merely as providing information pertinent to some specific embodiments that may fall within the scopes of the invention and the appended claims. Features from different embodiments may be employed in combination. In addition, other embodiments of the invention may also be devised which lie within the scopes of the invention and the appended claims. The scope of the invention is, therefore, indicated and limited only by the appended claims and their legal equivalents. All additions, deletions and modifications to the invention, as disclosed herein, that fall within the meaning and scopes of the claims are to be embraced by the claims.

What is claimed:

1. A device, comprising:
a first portion configured to be positioned adjacent a portion of a head of a user;
a second portion configured to be positioned adjacent a chest area of the user; and
a third portion extending between the first portion and the second portion and including an angular section.

2. The device of claim 1, the first portion comprising a u-shaped portion configured to contact a portion of a user's head.

3. The device of claim 1, the first portion comprising a u-shaped portion configured to contact the user's jaw region.

4. The device of claim 1, the first portion, the second portion, and the third portion comprising a unitary piece of material.

5. The device of claim 1, further comprising at least one of:
a first pad positioned on at least a portion of the first portion and configured to contact the user's jaw region; and
a second pad positioned on at least a portion of the second portion and configured to contact the user's chest region.

6. The device of claim 5, the first pad removably affixed to the first portion and comprising a neoprene material.

7. The device of claim 5, the second pad removably affixed to the second portion and comprising a neoprene material.

8. The device of claim 5, each of the first pad and the second pad comprising a neoprene material.

9. A device for stabilizing a head of a user in an upright position, comprising:
a chin support comprising a substantially u-shaped portion configured to contact a portion of a chin area of the user;
a first portion extending from the chin support;
a second portion extending from and at an angle to the first portion; and
a chest portion extending from the second portion and configured to contact a chest area of the user.

10. The device of claim 9, wherein the chin support is further configured to aid in causing a mouth of the user to be in a substantially closed position.

11. The device of claim 9, the chin support, the first portion, the second portion, and the chest portion comprising a unitary piece.

12. The device of claim 11, the unitary piece comprising plastic.

13. The device of claim 9, further comprising at least one of:
a strip positioned on at least a portion of the chin support and configured to provide at least one of padding and stability while the device is in use; and
another strip positioned on at least a portion of the chest portion and configured to provide at least one of padding and stability while the device is in use.

14. The device of claim 9, each of the strip and the another strip comprising neoprene.

15. The device of claim 9, each of the strip and the another strip comprising a padded strip.

16. The device of claim 9, wherein the strip is removably affixed to the chin support and the another strip is removably affixed to the chest portion.

17. A device, comprising:
a unitary piece of material configured to support a head of a user and including:
a first portion configured to be positioned adjacent a portion of the head of the user;
a second portion configured to be positioned adjacent a chest area of the user; and
a third portion extending between the first portion and the second portion and including an angular section.

18. The device of claim 17, the first portion comprising a u-shaped portion configured to contact a chin region of the user.

19. The device of claim 17, the second portion comprising a substantially planar surface configured to contact a chest region of the user.

20. The device of claim 17, wherein each of the first portion and the second portion comprise a pad positioned thereon and configured to provide padding to a user while the device is being used by the user.
